(19) United States
(12) Patent Application Publication
(10) Pub. No.: US 2021/0241882 A1
(43) Pub. Date: Aug. 5, 2021

(54) RADIOGRAPHIC IMAGE PROCESSING DEVICE, RADIOGRAPHY SYSTEM, RADIOGRAPHIC IMAGE PROCESSING METHOD AND RECORDING MEDIUM

(71) Applicant: KONICA MINOLTA, INC., Tokyo (JP)

(72) Inventor: Ryo WATANABE, Toyohashi-shi (JP)

(73) Assignee: KONICA MINOLTA, INC., Tokyo (JP)

(21) Appl. No.: 17/124,604

(22) Filed: Dec. 17, 2020

(30) Foreign Application Priority Data
Jan. 30, 2020 (JP) 2020-013256

(51) Int. Cl. G16H 30/20; G16H 30/40; G16H 40/67; G06F 21/62; A61B 6/00

(52) U.S. Cl. CPC G16H 30/20; G16H 30/40; A61B 6/563; G06F 21/6245; G16H 40/67

(57) ABSTRACT

A radiographic image processing device includes a communicator, a storage and a controller. The communicator communicates with an external device. The storage stores radiography data. The controller controls transmission of the radiography data and a request for confirming completion of save of the radiography data to the external device by the communicator. The controller sets a deletion time to delete personal information included in the transmitted radiography data after confirming completion of save of the radiography data. The controller deletes the personal information in the storage at the set deletion time.

### BACKGROUND

### 1. Technological Field

The present invention relates to a radiographic image processing device, a radiography system, a radiographic image processing method and a recording medium.

### 2. Description of the Related Art

Conventional systems in hospitals, medical cooperation facilities, etc. collect radiography data of medical images from radiography devices and keep them in databases, etc. Doctors and others comprehensively use radiography data for medical examination and the like. Recently, radiography devices such as an X-ray photography device has been miniaturized. The devices are brought to various places to take images. Radiography data is transferred through a wireless network to a storage device, such as database, via a radiographic image processing device, such as a portable computer.

There are standards for such medical images and communication thereof. In a medical field, generation, transfer processing and the like of radiographic image data are often carried out along the standard. JP2016-27893A discloses a technique related to issues such as data transfer from a processing device and a time to notify completion when image data is transferred in accordance with DICOM (Digital Imaging and Communications in Medicine) standard. Image data is generated and transmitted by a radiography device based on a request for examination by the HIS/RIS (Hospital Information System/Radiology Information System). Image data and various additional data related to the image data are sent to a storage device, and a response for entrusted save which indicates that the data have been stored in the storage device is obtained. The data can be deleted when there is a lack of storage space in the processing device.

The additional data related to radiographic image data includes information that identifies a patient who is a subject of radiography. Radiography devices are often shared by multiple technicians, etc. Therefore, personal information could be leaked if a radiography device keeps data without deleting it until a storage space is exhausted. This is a problem.

### SUMMARY

An object of the invention is to provide a radiographic image processing device, a radiography system, a radiographic image processing method and a recording medium that manage personal information more properly.

To achieve at least one of the abovementioned objects, according to an aspect of the present invention, a radiographic image processing device includes:
a communicator that communicates with an external device;
a storage that stores radiography data; and
a controller,
wherein
the controller controls transmission of the radiography data and a request for confirming completion of save of the radiography data to the external device by the communicator,
the controller sets a deletion time to delete personal information included in the transmitted radiography data after confirming completion of save of the radiography data, and
the controller deletes the personal information in the storage at the set deletion time.

According to another aspect of the present invention, a radiography system includes:
the radiographic image processing device; and
a radiography device that takes radiographic images.

According to still another aspect of the present invention, a radiographic image processing method for a radiographic image processing device is provided,
the device including:
   a communicator that communicates with an external device; and
   a storage that stores radiography data, and
the method including:
   controlling transmission of the radiography data and a request for confirming completion of save of the radiography data to the external device by the communicator,
   setting a deletion time to delete personal information included in the transmitted radiography data after confirming completion of save of the radiography data, and
   deleting the personal information in the storage at the set deletion time.

According to still another aspect of the present invention, a non-transitory computer readable medium stores a program that makes a computer of a radiographic image processing device perform processing,
the device including:
   a communicator that communicates with an external device; and
   a storage that stores radiography data, and
the processing including:
   controlling transmission of the radiography data and a request for confirming completion of save of the radiography data to the external device by the communicator,
   setting a deletion time to delete personal information included in the transmitted radiography data after confirming completion of save of the radiography data, and
   deleting the personal information in the storage at the set deletion time.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 shows the overall structure of a medical image management system.

FIG. 2 is a block diagram showing functional configuration of a radiography control device.

FIG. 3A and FIG. 3B are sequence diagrams showing a flow of processing related to radiography data between the radiography control device and an image management device.

FIG. 4 is a flow chart showing control procedure in radiography control processing.

FIG. 5 is a flowchart showing control procedure in personal information deletion processing.

FIG. 6 is a flowchart showing Modification 1 of the control procedure in the radiography control processing.

FIG. 7 is a flowchart showing Modification 2 of the control procedure in the radiography control processing.

FIG. 8 is a flowchart showing Modification 3 of the control procedure in the radiography control processing.

FIG. 9 is a flow chart showing control procedure in display control processing.

FIG. 10 is a flowchart showing Modification 4 of the control procedure in the radiography control processing.

### DETAILED DESCRIPTION OF EMBODIMENTS

Hereinafter, one or more embodiments of the present invention will be described with reference to the drawings. However, the scope of the invention is not limited to the disclosed embodiments.

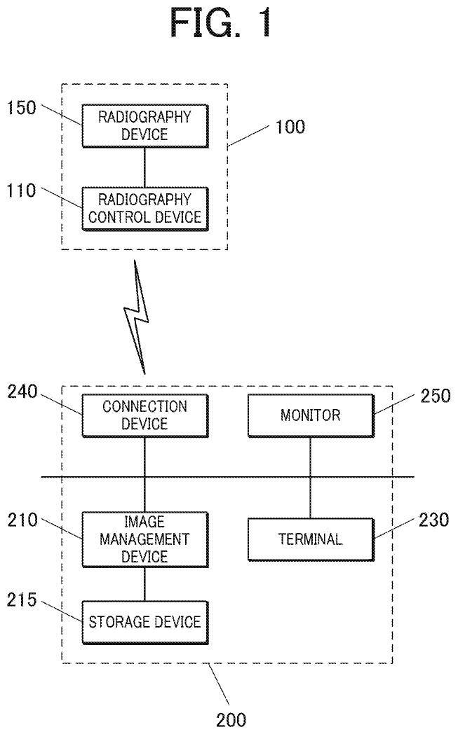

FIG. 1 shows the overall structure of a medical image management system 1 according to the embodiment.

The medical image management system 1 includes a radiography system 100 and an image save communication system 200.

The radiography system 100 includes a radiography control device 110 (radiographic image processing device) and a radiography device 150. The radiography device 150 emits and detects radiation, such as an X-ray, based on control of the radiography control device 110 to take images of a patient's examination part. The detection result is obtained as digital data, and radiographic image data is obtained.

The radiography control device 110 controls the radiography device 150 for radiography. The radiography control device 110 obtains radiographic image data and generates radiography data with information such as radiography information and personal information included in an examination request. The personal information is information which identifies a patient and which at least includes a patient's name and a patient ID. In addition to them, the personal information may include information such as age, gender and department. The radiography control device 110 communicates with the image save communication system 200 via a wireless LAN (Local Area Network) or the like. The radiography control device 110 may be a computer such as a common PC (Personal Computer) or may be a dedicated computer terminal.

The radiography system 100 of the embodiment is easily carried and moved. The radiography system 100 is brought to patient's rooms, etc., to perform various processing such as radiography. In the embodiment, the radiography device 150 and the radiography control device 110 are connected with a cable. However, they may be wirelessly connected. Combination of the radiography device 150 and the radiography control device 110 may be specified. Alternatively, one radiography control device 110 may be combined with various radiography devices 150. Individual radiography device 150 may be prepared for each part to be photographed or the like. Alternatively, the radiography device 150 may have various replaceable parts for various parts to be photographed.

The image save communication system 200 is, for example, a system standardized as PACS (Picture Archiving and Communication System). The image save communication system 200 includes an image management device 210, a storage device 215, a terminal 230, a connection device 240, and a monitor 250.

The image management device 210 obtains radiography data from various radiography systems 100. The image management device 210 makes the storage device 215 store the radiography data to manage the data. The image management device 210 allows the terminal 230 to access (view, analyze, etc.) the radiography data in response to a request from the terminal 230, etc.

The storage device 215 stores and keeps the radiography data as described above. In the embodiment, the storage device 215 is externally attached to the image management device 210. Alternatively, the storage device 215 may be a built-in storage inside the image management device 210 or may be a cloud server or a database server directly connected to a network.

The terminal 230 includes a computer which a doctor or the like uses for displaying taken images and the like in diagnosis. Although only one terminal 230 is provided in the embodiment, more than one terminal 230 may be connected or connectable to the image save communication system 200.

The connection device 240 is, for example, a router for a wireless LAN and is an access point for communicating with the radiography system 100. The connection device 240 may be connected with multiple radiography systems 100.

The monitor 250 obtains status data of various processing from the image management device 210 and the like and monitors whether processing is properly performed. The monitor 250 monitors, for example, whether the whole processing including a series of operations is completed normally. If a part of processing is not performed or when processing is interrupted, the monitor 250 outputs alert information to the image management device 210, other display devices for monitoring, etc.

The image management device 210 and the storage device 215 are external devices in the embodiment.

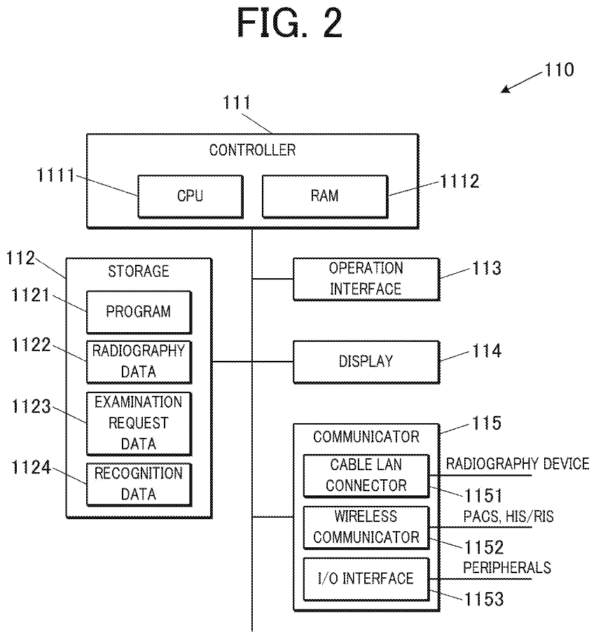

FIG. 2 is a block diagram showing functional configuration of the radiography control device 110. The radiography control device 110 includes a controller 111, a storage 112, an operation interface 113, a display 114, and a communicator 115.

The controller 111 comprehensively controls operations of parts of the radiography control device 110. The controller 111 is a hardware processor including a CPU 1111 (Central Processing Unit) and RAM 1112 (Random Access Memory). The CPU 1111 performs arithmetic operations and various control operations. The RAM 1112 provides the CPU 1111 with working memory space and stores temporary data.

The storage 112 is a non-volatile memory and includes, for example, an HDD (Hard Disk Drive) and/or flash memory. The storage 112 stores a program 1121, radiography data 1122, examination request data 1123, recognition data 1124, and the like.

The program 1121 includes control programs for various processing such as radiography control processing and personal information deletion processing described below. The CPU1111 reads and executes these control programs.

The radiography data 1122 includes the radiographic image data taken by the radiography device 150 and additional data related to the radiographic image data, such as examination ID, patient ID, patient name, age, gender, shooting date and time, and shooting condition. The radiography data 1122 may include setting data as to whether the radiographic image data included in the radiography data 1122 may be deleted, whether there is a setting (recognition setting) of restricting disclosure of personal information or the entire radiography data, etc.

The examination request data 1123 is data of a request for radiography examination which is obtained from a HIS/RIS or the like. The examination request data 1123 includes information such as examination ID, patient ID, patient name, age, sex, date and time of radiography reservation, and a part to be photographed.

The recognition data 1124 is used for encrypting personal information included in radiography data and the like. The recognition data 1124 is set to relate to the radiography data 1122 for which disclosure restriction is set, and then stored. Contents of the recognition data 1124 may not be managed collectively. They may be respectively related to the radiography data 1122 or stored respectively in pieces of data.

The operation interface 113 receives input operation from outside, such as an examiner, etc. who is a user of the radiography control device 110. The operation interface 113 outputs the input operation to the controller 111 as an input signal. The operation interface 113 includes some or all of a touch panel piled on the display 114, a keyboard that receives key operation, a pointing device such as a mouse, various switch buttons, and the like. Apart of the operation input device may be connected to a USB (Universal Serial Bus) port via an I/O interface 1153 described below.

The display 114 displays various contents on a display screen based on control of the controller 111. The display screen is, for example, a color liquid crystal display screen. However, the display screen is not limited to this. Contents to be displayed include contents of radiography data, i.e., radiographic image data and additional information data. The display 114 may include an indicator and/or an alert lamp such as an LED (Light Emitting Diode).

The communicator 115 controls transmission of data and communication with external devices and peripherals. The communicator 115 includes, for example, a cable LAN connector 1151, a wireless communicator 1152, and an I/O interface 1153 (external output). The cable LAN connector 1151 may be a network card or the like that controls cable connection with a network via a LAN. The wireless communicator 1152 controls wireless communication via a wireless LAN or the like. The I/O interface 1153 includes a connection terminal for USB connection, etc. for transmission of data to/from peripherals. In the embodiment, a cable LAN connector 1151 is connected to the radiography device 150. The wireless communicator 1152 is connected to the PACS200 (external devices including the image management device 210), a HIS/RIS (not shown), etc. Peripherals connected to the I/O interface 1153 may include storage devices such as an external HDD and flash memory, and/or a disk drive for reading and writing from/to a recording medium (especially for writing and outputting radiography data) such as a DVD and a Blu-ray (registered trademark) disc, in addition to devices for operation such as the pointing device described above. The peripherals may be wirelessly connected to the radiography device 150 or be connected by cable to devices of the PACS200 such as the image management device 210.

Next, a flow of radiography data in the medical image management system 1 according to the embodiment will be explained. The radiography device 150 takes a radiographic image, and the radiography control device 110 generates and obtains radiography data. The radiography data is transmitted to the PACS200 to be saved and stored. After that, the radiography data kept in the radiography control device 110 is deleted to save space for storing subsequent radiography data. For the purpose of redisplay or the like, some radiography data may be kept without being deleted. Designation as data not to be deleted may be performed in response to acceptance of predetermined input operation in the operation interface 113 at any time before radiography data is deleted. Set designation as data not to be deleted may be canceled if the designation becomes unnecessary.

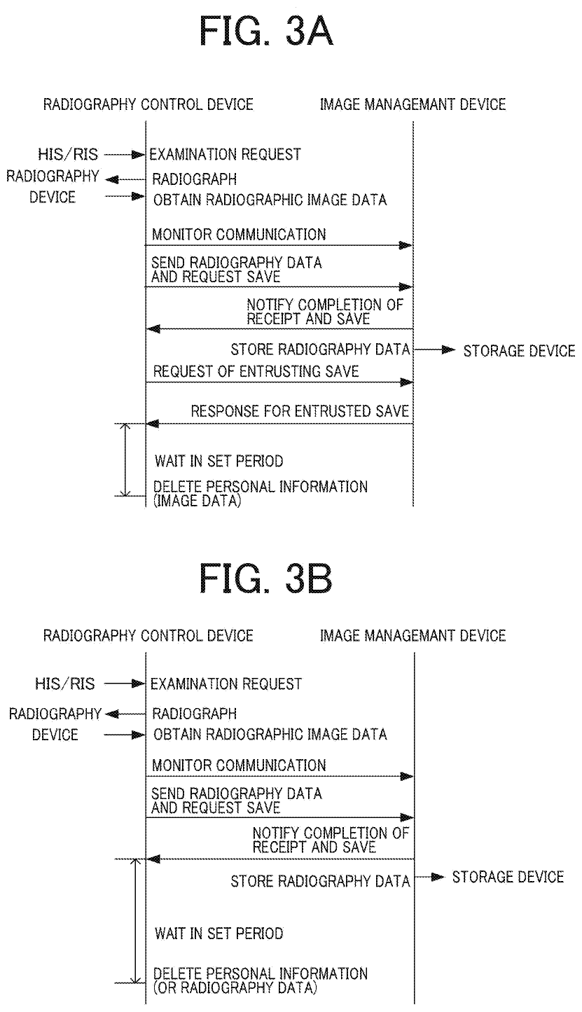

FIG. 3A and FIG. 3B are sequence diagrams showing a flow of processing related to radiography data between the radiography control device 110 and the image management device 210.

As shown in FIG. 3A, between the image management device 210 and the radiography control device 110 which is based on DICOM standard, the radiography control device 110 first receives an examination request from the HIS/RIS and operates the radiography device 150 to take images based on the examination request. The radiography control device 110 obtains radiographic image data and adds additional information to generate radiography data. Some initial processing may be performed on the radiographic image data. The initial processing includes, for example, processing of extracting only a target area from a widely captured image, processing of adjusting resolution, contrast, and the like, processing of rotating an image, and processing of reducing noise.

After the radiography control device 110 obtains the radiography data, the controller 111 of the radiography control device 110 monitors communication condition (condition of wireless communication) between the radiography control device 110 and the image management device 210). The controller 111 controls the radiography control device 110 to begin transmitting radiography data to the image management device 210 when connection is in good condition. At the same time, the radiography control device 110 sends, to the image management device 210, a request for reply that confirms completion of save of the radiography data. The image management device 210 receives this radiography data and the request, and stores and accumulates the radiography data in the storage. Data may be transferred by the packet. If the communication is temporarily disconnected during transfer, the transfer is interrupted. The remaining packet data may be transmitted after communication resumes. Upon completion of receipt and storage, the image management device 210 transmits a notification of successful completion of save to the radiography control device 110. The radiography data stored in the image management device 210 is transferred to the storage device 215 to be saved and stored.

After the radiography control device 110 completes confirmation of completion of save by receiving the notification of completion of save, i.e. notification of successful save, the radiography control device 110 transmits a request of entrusting save to the image management device 210. The request of entrusting save is processing that confirms completion of transfer of radiography data to the storage device 215 and storage of the data. In the embodiment, the request of entrusting save is transmitted in accordance with DICOM standard. After the image management device 210 completes transfer of radiography data to the storage device 215 and storage of the data, the image management device 210 transmits response for entrusted save which indicates successful storage to the radiography control device 110.

In the embodiment, the request for confirmation of completion of save includes at least a request for notification of completion of save. The request for confirmation of completion of save may also include the request of entrusting save.

After the radiography control device 110 receives the response for entrusted save, the radiography control device 110 waits until a set period passes, and deletes the radiography data when the set period passes. In a case in which the radiographic image data is designated as data not to be deleted before a deletion time, the radiography control device 110 deletes not the entire radiography data but only personal information data included in the radiography data. To meet requirements for security, personal information data may be deleted as soon as the set period passes. However, personal information data may not be deleted as soon as the set period passes. In a case in which the deletion might cause a trouble or the like after the set period passes as described below, the radiography control device 110 may delete the data after waiting further until a predetermined condition is met, the condition being for sufficiently reducing likelihood of trouble. It is different from conventional technique in which a time to delete is determined based on an empty space of the storage 112 irrespective of a set period. In addition to requirements for security, an upper limit of the set period may be determined to more stably save empty space of the storage 112.

Some combinations of the radiography control device 110 and the image management device 210 may not correspond to processing of the request of entrusting save and the response for entrusted save. In that case, these communications may be omitted as shown in FIG. 3B. The radiography control device 110 proceeds to waiting in the set period when it receives a notification of successful save.

Thereby, the radiography control device 110 normally deletes radiography data before using up storage space for the radiography data 1122 in the storage 112.

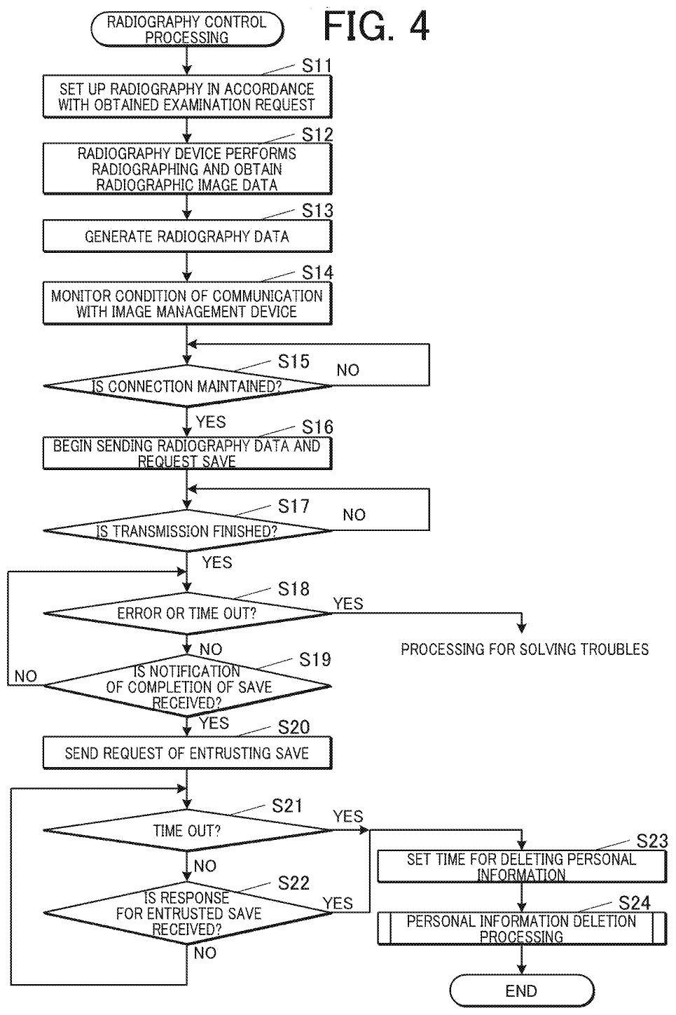

FIG. 4 is a flow chart showing control procedure of the controller 111 in the radiography control processing performed by the radiography control device 110 according to the embodiment. The radiography control processing is started when, for example, an examination request obtained from external devices, especially the HIS/RIS, is added to the examination request data 1123.

When the radiography control processing begins, the controller 111 (CPU1111) performs a radiographic setting according to radiography contents included in an obtained examination request (Step S11). The controller 111 makes the radiography device 150 radiograph an examination object in accordance with the radiographic setting to obtain radiographic image data (Step S12). The controller 111 combines the radiographic image data with additional information, such as personal information and radiography conditions, to generate radiography data (Step S13).

The controller 111 monitors communication condition between the radiography control device 110 and the image management device 210 (Step S14). The controller 111 determines whether connection is maintained (Step S15). In a case in which the controller 111 determines that connection is not maintained (disconnected or intermittently disconnected) (“NO” in step S15), the controller 111 repeats processing of Step S14.

In a case in which the controller 111 determines that connection is maintained (“YES” in Step S15), the controller 111 begins transmission of radiography data to the image management device 210 (Step S16). The controller 111 may monitor communication condition during transmission to control transmission of packet data. The controller 111 determines whether transmission of radiography data is completed (Step S17). In a case in which the controller 111 determines that transmission of radiography data is not completed (“NO” in step S17), the controller 111 repeats processing of step S17.

In a case in which the controller 111 determines that transmission is completed (“YES” in Step S17), the controller 111 determines whether a reply of failure in save is received or whether a predetermined timeout period has passed since completion of transmission (or start of transmission) to bring timeout (Step S18). The timeout period may be the same as, or longer than, a period from the time at which the image management device 210 attempts to send a notification of completion of save to the time at which transmission ends without being completed due to communication condition or the like. In a case in which the controller 111 determines that the reply of failure in save is received or the timeout is brought about (“YES” in Step S18), the controller 111 proceeds to various processing for solving troubles. The processing for solving troubles may include, for example, processing of simply re-sending radiography data, or operation of notifying unusual operation of the image management device 210 and/or the storage device 215.

In a case in which the controller 111 determines that the reply of failure in save is not received and the timeout is not brought about (“NO” in Step S18), the controller 111 determines whether the notification of completion of save is received (Step S19). In a case in which the controller 111 determines that the notification of completion of save is not received (“NO” in Step S19), processing of the controller 111 returns to Step S18.

In a case in which the controller 111 determines that the notification of completion of save is received (“YES” in Step S19), the controller 111 transmits the request of entrusting save to the image management device 210 (Step S20). In Step S21, the controller 111 determines whether a predetermined timeout period has passed since the request of entrusting save is transmitted to bring timeout (Step S21). The timeout period in this processing may be or may not be the same as the timeout period in Step S18. In a case in which the controller 111 determines that the timeout is not brought about (“NO” in Step S21), the controller 111 determines whether the response for entrusted save, which indicates storage of radiography data in the storage device 215, is received (Step S22). In a case in which the controller 111 determines that the response for entrusted save is not received (“NO” in Step S22), processing of the controller 111 returns to Step S21.

In a case in which the controller 111 determines that the response for entrusted save is received (“YES” in Step S22), the controller 111 sets a deletion time (standby period) to delete personal information in transmitted radiography data (Step S23). To set the deletion time, such as date and time for deletion, the controller 111 may make the display 114 display a request for input of a separate setting for each radiography data. Setting up is performed in accordance with contents of the input operation received by the operation interface 113. The contents received through the input operation may be a standby period. Alternatively, date and time may be specified directly. In a case in which there is no input operation or no request for input operation, the deletion time may be set based on a common standby period stored in advance, current date and time, etc. Alternatively, the standby period may be set in advance for each radiography data. The standby period can be any time equal to or more than “0” (predetermined shortest time limit). Alternatively, the standby period may not include “0” and may be equal to or greater than a minimum unit time that can be set (e.g., 1 min) (longer than the predetermined shortest time limit). The standby period may be set at, for example, a value equal to or less than a period in which the oldest radiography data 1122 is kept, the oldest radiography data 1122 being the oldest piece of data among multiple pieces of radiography data currently kept except those designated as data not to be deleted. The controller 111 invokes and executes personal information deletion processing (Step S24). The controller 111 then ends the radiography control processing.

In a case in which the controller 111 determines that the timeout is brought about in the determination processing of Step S21 (“YES” in Step S21), processing of the controller 111 proceeds to Step S23. Although no response is sent for the request of entrusting save and receipt of notification has failed, it is confirmed that the image management device 210 has already retained the radiography data. Therefore, it is not usually assumed that data is not stored in the storage device 215, such as data server, including a redundant system. Thus, even when receipt of response for entrusted save has failed, confirmation of completion of save can be regarded as normally finished, and processing may proceed to a setting for deleting personal information (radiography data). In a case in which a reply that storage of radiography data in the storage device 215 has failed is received, processing proceeds to processing for solving troubles like a case in which completion of save has failed.

Processing of Step S24 may not be continuously performed as a part of the radiography control processing. For example, in a case in which a trigger signal is obtained in response to an interruption command or the like at date and time when waiting ends, the processing of Step S24 may be separately invoked and performed.

In the above processing, processing of Steps S14 to S22 corresponds to a step of controlling communication (means of controlling communication) in the radiographic image processing method (program) according to the embodiment. Processing of Step S23 corresponds to a setting step (setting means). Processing of Step S24 corresponds to a deletion step (deletion means).

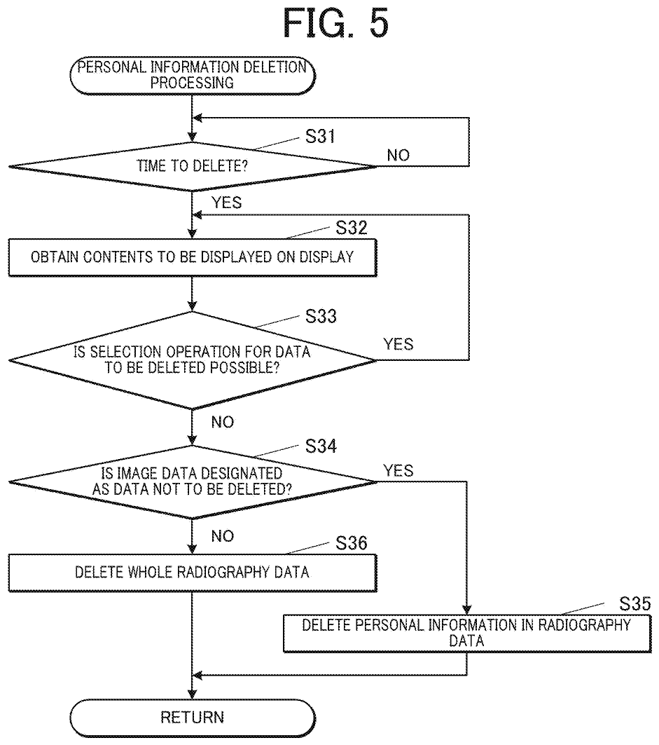

FIG. 5 is a flow chart showing control procedure in personal information deletion processing invoked in the radiography control processing.

When the personal information deletion processing is invoked, the controller 111 determines whether it is the deletion time for radiography data to be deleted (Step S31). In a case in which the controller 111 determines that it is not the deletion time (“NO” in Step S31), the controller 111 repeats processing of step S31. In a case in which the controller 111 determines that it is the deletion time (“YES” in Step S31), the controller 111 obtains contents to be displayed on the display 114 (step S32). The controller 111 determines whether operation of selecting some processing related to contents (personal information) of radiography data to be deleted is possible (Step S33) based on input operation to the operation interface 113 (touch panel). If options for some operation concerning radiography data to be deleted or an input window for invoking the radiography data appears on the display screen, selection operation is possible. In a case in which the controller 111 determines that the selection operation is possible (“YES” in Step S33), processing of the controller 111 returns to Step S32.

In a case in which the controller 111 determines that the selection operation is not possible, that is, a case in which neither the option nor the input window for invoking appears, or a case in which selection or the like of the options is not possible, for example, display contents on the display screen is being switched (“NO” in Step S33), the controller 111 determines whether any piece of radiographic image data is designated as data not to be deleted (Step S34). In a case in which the controller 111 determines that a piece of the radiographic image data is designated (“YES” in Step S34), the controller 111 deletes only personal information in the radiography data (Step S35). The controller 111 then ends the personal information deletion processing and returns to the radiography control processing.

In a case in which the controller 111 determines that all the pieces of the radiographic image data is not designated as data not to be deleted, the controller 111 deletes all the pieces of radiography data including a piece of radiographic image data corresponding to personal information to be deleted (Step S36). The controller 111 then ends the personal information deletion processing and returns to radiography control processing.

In the case in which the personal information deletion processing is started from the interruption processing at the deletion time as described above, the processing of Step S31 may be omitted.

Thus, in a situation where the operation interface 113 can select or input radiography data to be deleted based on the display contents, a display position may be unintentionally shifted, or display contents may be switched to other contents if deletion is performed during input operation. It causes errors in operation. In the situation where such selection or input is possible, the controller 111 may further wait without deleting the radiography data (personal information) and perform the deletion processing when selection or input becomes impossible. In a case in which contents to be displayed after switching relates to radiography data to be deleted, if the contents are already read from the storage 112 and deployed in the RAM 1112, display may be performed based on data in the RAM 1112. Alternatively, display may be controlled such that processing cannot proceed from the display contents to be updated to other display contents in the radiography data.

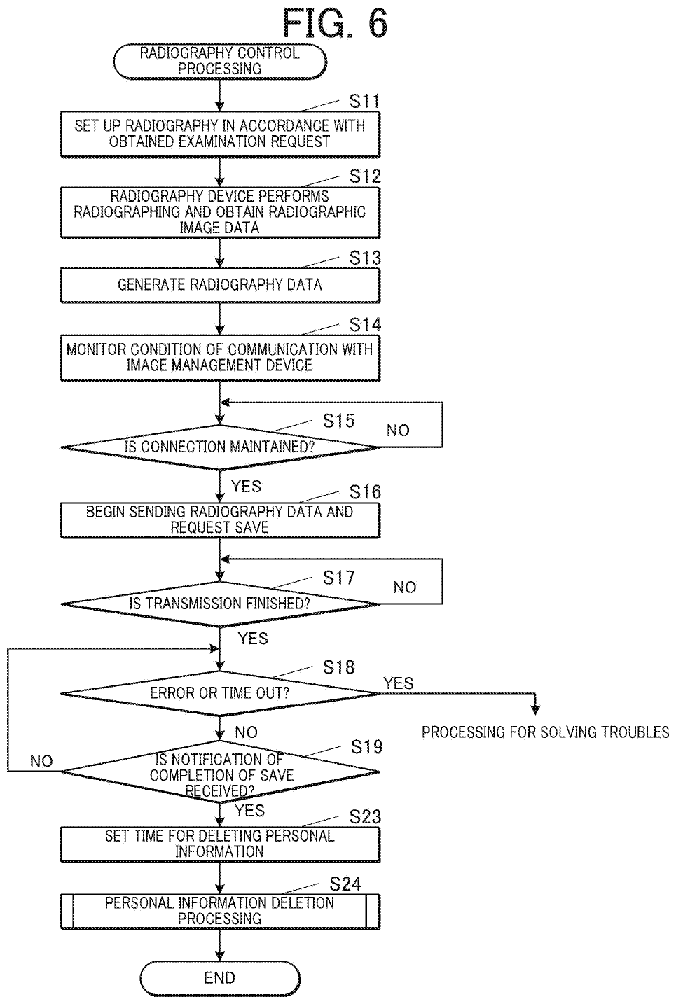

FIG. 6 is a flowchart showing Modification 1 of control procedure in the radiography control processing.

The radiography control processing of Modification 1 corresponds to processing in FIG. 3B, and is different from the radiography control processing in FIG. 4 in that processing of Steps S20 to S22 is deleted. When processing proceeds to “YES” in Step S19, processing shifts to Step S23. Except for that, processing is the same. The same numerals are given and details are omitted.

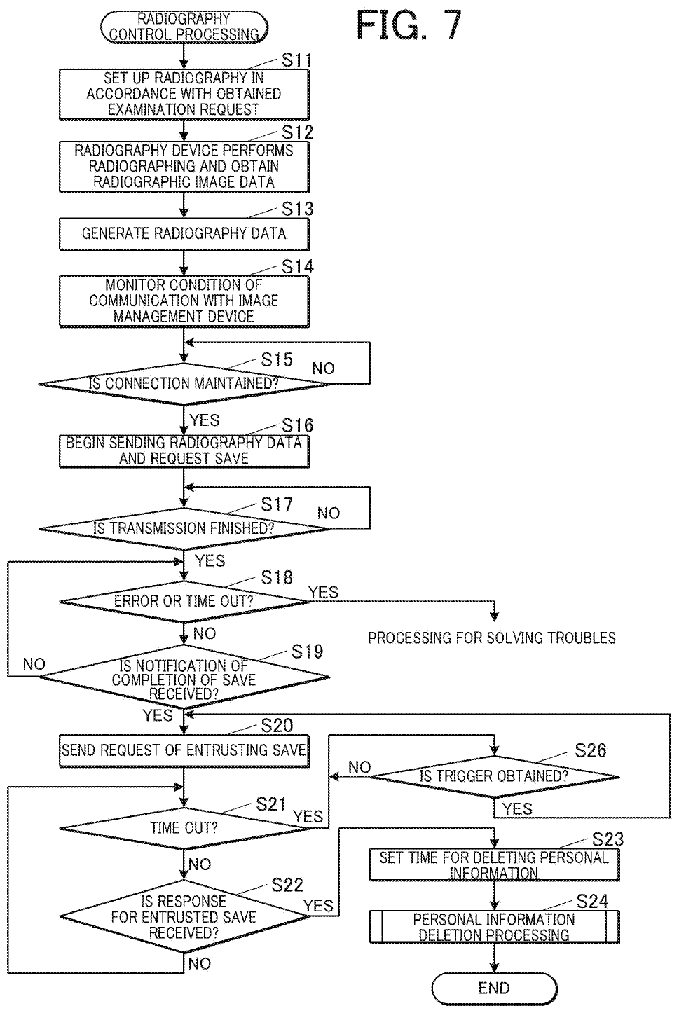

FIG. 7 is a flowchart showing Modification 2 of control procedure in the radiography control processing.

The radiography control processing of Modification 2 is different from the radiography control processing in FIG. 4 in that processing of Step S26 is added. Except for that, processing is the same. The same numerals are given to the same processing, and details are omitted.

In the radiography control processing of Modification 2, when the response for entrusted save is not obtained within the timeout period, processing does not immediately shift to a setting for deleting personal information. The request of entrusting save is sent again in response to a predetermined trigger. The trigger is, for example, input from the outside via the communicator 115. In the embodiment, the trigger is input from the monitor 250. For example, after the monitor 250 detects that the image management device 210 receives the request of entrusting save, the monitor 250 monitors radiography data for which the response for entrusted save from the image management device 210 has not reached the radiography control device 110. The monitor 250 outputs periodically, for example, at predetermined time intervals, a request for resending information of the radiography data and the request of entrusting save (command for resending) to the radiography control device 110 which sent the radiography data. When the radiography control device 110 obtains the information, the radiography control device 110 determines to resend the request of entrusting save for the radiography data included in the information one after another.

Alternatively, the controller 111 may generate and keep a list of radiography data for which the response for entrusted save is not received after the request of entrusting save is sent so that timeout is brought about. The controller 111 may then select radiography data for which the response for entrusted save (notification) is not received yet from the list, and make the radiography control device 110 resend the request of entrusting save one after another.

In the radiography control processing in FIG. 7, in a case in which the controller 111 determines that timeout is brought about in processing of Step S21 (“YES” in Step S21), the controller 111 may perform processing of adding an ID, etc. of radiography data to the above list in the storage 112 or processing of updating the ID, etc. The controller 111 then determines whether the predetermined trigger is obtained (Step S26). As described above, the trigger is, for example, receipt of list information of radiography data from the monitor 250 or reading of a list of radiography data stored in the storage 112, the radiography data being the one for which the response for entrusted is not received.

In a case in which the trigger is not obtained (“NO” in Step S26), the controller 111 repeats processing in Step S26. In a case in which the trigger is obtained (“YES” in step S26), processing of the controller 111 returns to Step S20.

In a case in which the storage 112 stores and keeps the list, the controller 111 may perform processing of deleting an ID of radiography data stored in the list when processing proceeds to “YES” in Step S22.

This decision processing of trigger obtainment may also be separated from the radiography control processing by interruption processing. In that case, in a case in which timeout is brought about in processing of Step S21, the radiography control processing is once ended. Then, when the above information is obtained from the monitor 250, processing in Steps S20 to S24 is activated and performed one after another for radiography data included in the information.

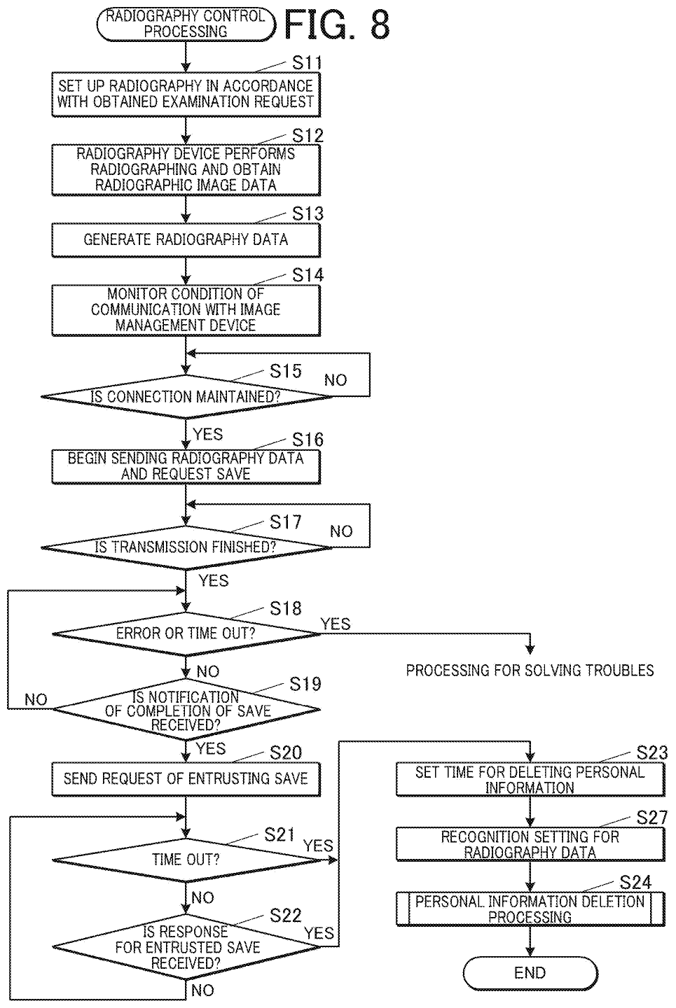

FIG. 8 is a flowchart showing Modification 3 of control procedure in the radiography control processing.

The radiography control processing of Modification 3 is different from the radiography control processing in FIG. 4 in that processing of Step S27 is added. Except for that, processing is the same. The same numerals are given to the same processing, and details are omitted.

In the radiography control processing of Modification 3, a recognition setting is performed to restrict disclosure on at least personal information in radiography data kept in the storage 112 until the radiography data is deleted.

After the radiography data is transmitted, in this Modification, after processing of Step S23, the controller 111 makes a recognition setting for radiography data (Step S27). The radiography data 1122 is often transmitted as soon as radiography is performed. A radiographer is unlikely to be changed to another person before transmission. It is troublesome if radiography data is transmitted after disclosure is restricted by the recognition setting. Therefore, restriction of disclosure is set after transmission. It provides secure management of personal information and easy operation at the same time. The controller 111 requests input of recognition information, e.g., a password, a passphrase, a PIN, etc., for this radiography data on the display screen. At least personal information in the radiography data is encrypted according to the inputted recognition information. In this modification, the entire data including the radiographic image data is encrypted. Then, processing of the controller 111 proceeds to Step S24.

Processing of Step S27 may not be performed after Step S23. In a case in which processing of Step S27 is performed prior to processing of Step S16, data may be once decrypted based on the recognition information and then transmitted in accordance with the DICOM standard in processing of Step S16. Data transmission of the PACS200 is standardized. To transmit data, data is once decrypted in accordance with the standard and then output. It improves management condition of personal information easily and effectively. The medical image management system 1 does not need to be changed entirely. Partial modification is sufficient.

In a case in which disclosure of personal information only is restricted, when the number of radiographed patients is small, an object of radiographic image data is easily identified by others though disclosure of personal information is restricted. In that case, disclosure of radiographic image data should be restricted so that personal information is managed more properly.

In a case in which disclosure is restricted, radiography data needs to be decrypted before the radiography control device 110 displays the radiography data. Identification information other than personal information, such as an examination ID, a radiography ID, radiography date and time, may not be encrypted so that a user can select radiography data to be displayed. Alternatively, an encrypted file name, a header of an encrypted file, and the like may separately include such information so that an examination ID, a radiography ID, radiography date and time, and the like of the encrypted data are specified.

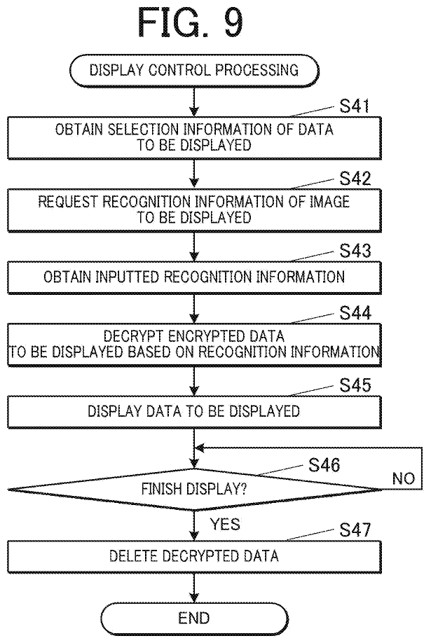

FIG. 9 is a flow chart showing control procedure of the controlling display control processing by controller 111.

In a case in which the operation interface 113 or the like receives (reads out) a command to display encrypted radiography data, the controller 111 obtains identification information of the data to be displayed (Step S41). The controller 111 makes the display 114 display an input window for the recognition information to request input of the recognition information (Step S42). The controller 111 receives input of the recognition information in the input window and obtains the inputted recognition information (Step S43).

In a case in which the recognition information is correct, the controller 111 decrypts radiography data to be displayed based on the recognition information (Step S44). The controller 111 makes the display 114 display the decrypted radiography data in a requested format (Step S45). The format may be changed as appropriate in accordance with operation received by the operation interface 113. The controller 111 updates display contents in response to the changed format.

The controller 111 determines whether a command to terminate display is obtained (Step S46). The command to terminate display is outputted in response to predetermined input operation or the like received by the operation interface 113. The command to terminate display may be outputted also when processing of updating display is not received and a predetermined period passes so that timeout is brought about. In a case in which the controller 111 determines that the command is not obtained (“NO” in Step S46), the controller 111 repeats processing of Step S46. In a case in which the controller 111 determines that the command is obtained (“YES” in Step S46), the controller 111 ends display and deletes the decrypted data (Step S47). In a case in which encryption is performed in a manner in which encrypted data is deleted at the time of decryption, the controller 111 may encrypt decrypted data based on the same recognition information again. The controller 111 then ends the display control processing.

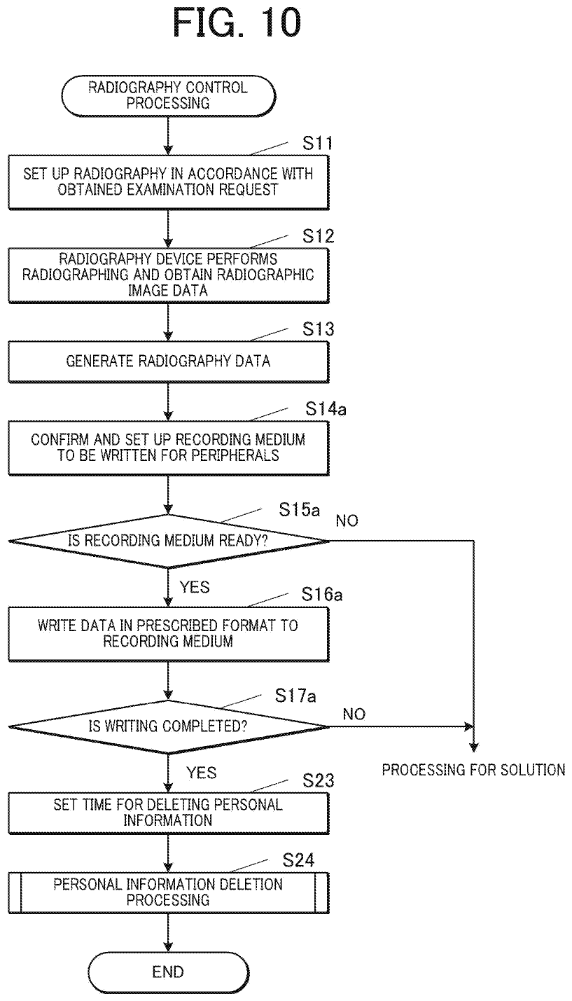

FIG. 10 is a flowchart showing Modification 4 of control procedure of the radiography control processing.

The radiography control processing of Modification 4 shows control of writing and outputting in a prescribed format to a recording medium, e.g., a DVD, a Blu-ray disc, a magnetic tape, etc., of peripherals connected to the I/O interface 1153 instead of transmitting radiography data to the image management device 210. In this radiography control processing, processing of Steps S14 to S17 in the radiography control processing of Modification 1 in FIG. 6 is replaced by processing of Steps S14a to S17a, respectively. In addition, processing of Steps S18 and S19 are omitted. Except for them, processing is the same. The numerals are given to the same processing, and details are omitted.

When radiography data is generated in processing of Step S13, the controller 111 confirms whether a writing device for a recording medium (e.g., a portable recording medium such as an optical disc) is connected to the I/O interface 1153 as one of peripherals. The controller 111 also checks whether writing to the recording medium is possible. The controller 111 performs processing such as initialization as required (Step S14a). The controller 111 determines whether writing to the recording medium is possible (Step S15a). In a case in which the controller 111 determines that writing is not possible (“NO” in Step S15a), the controller 111 proceeds to processing for solving problems. In a case in which only insertion, replacement, etc. of the recording medium is required, processing may return to Step S14a.

In a case in which the controller 111 determines that writing to the recording medium is possible (“YES” in Step S15a), the controller 111 writes radiography data to the recording medium in the prescribed format (Step S16a). The controller 111 determines whether writing is normally completed (Step 17a). In a case in which the controller 111 determines that writing is not normally completed (“NO” in Step S17a), processing of the controller 111 proceeds to processing for solving problems.

In a case in which the controller 111 determines that the writing and output is normally completed (“YES” in Step S17a), processing of the controller 111 proceeds to Step S23. Then, the controller 111 deletes personal information when deletion is permitted after the set standby period passes.

Personal information may be similarly managed in a case in which radiography data 1122 is directly written to an optical disc, etc. like the radiography control device 110 in Modification 4. It more reliably prevents personal information from being leaked.

As described above, the radiography control device 110 according to the embodiment includes:
a communicator 115 that communicates with the image management device 210 which is an external device;
a storage 112 that stores radiography data 1122; and
the controller 111.

The controller 111 controls transmission of radiography data 1122 to the image management device 210 and of a request for confirming completion of save of the radiography data 1122 (request for save and request of entrusting save), the transmission being performed by the communicator 115. The controller 111 confirms completion of save of transmitted radiography data 1122, and then set deletion time to delete personal information in the radiography data 1122. The controller 111 deletes the personal information in the storage 112 at the deletion time.

Thus, at least personal information is deleted after a time appropriately set in accordance with reuse plan, etc. and before a period in which radiography data 1122 can usually be kept in the radiography control device 110 passes. Thereby, personal information is managed more properly. Personal information is deleted after it is confirmed that radiography data is at least transferred and kept in the PACS200. Therefore, data is properly managed without being accidentally deleted.

The radiography control device 110 includes the operation interface 113. The controller 111 sets deletion time based on input operation received by the operation interface 113.

In some examinations, radiographic image data of one time is compared with radiographic image data of another time. Therefore, instead of simply deleting radiography data right after the radiography data is transferred, a standby period of radiography data is set based on input operation in accordance with radiography. Accordingly, radiography data including necessary personal information is flexibly kept and utilized.

The standby period from the time when confirmation of completion of save is finished to the deletion time is longer than a predetermined shortest time limit. Thus, personal information is not deleted right after radiography data is transferred. As a result, the personal information can be referenced and utilized in later examinations, etc.

After the deletion time, the controller 111 deletes personal information when a predetermined condition is met. In a case in which personal information should not be deleted at the deletion time for reasons of operation or user's works, the standby period can be slightly prolonged. It appropriately reduces adverse effects on other processing and users' tasks, and improves processing performance, operability, etc.

The radiography control device 110 includes a display 114 that displays contents of radiography data 1122. The controller 111 determines, as the predetermined condition, whether display or processing of contents of the radiography data 1122 related to personal information to be deleted can be changed or selected. If neither change nor selection is possible, the controller 111 deletes the personal information. A bad example in operation or tasks is a case in which radiography data is deleted while a user is performing select operation. It causes deficiencies. For example, information corresponding to an image which is selected is not displayed, or other radiography data is displayed to cause confusion or misunderstanding. To avoid this, the time to delete personal information is adjusted.

In a case in which neither change nor selection is possible, the controller 111 adds a period during which display contents of the display 114 is switched. User's operation is not received while display contents are switched. It prevents user's operation from being wasted and prevents misunderstanding of display contents.

The controller 111 selects a piece of radiography data 1122 for which the response for entrusted save is not obtained from the image management device 210 among pieces of radiography data 1122 stored in the storage 112. The controller 111 transmits the request of entrusting save for the selected piece of radiography data 1122 again to the image management device 210. Thus, radiography data for which final storage in the storage device 215 is not confirmed is listed. Failure in confirmation is caused by deterioration of communication condition, etc., for example, a temporary communication break due to movement of the radiography control device 110. The controller 111 automatically and periodically checks storage of data. It saves users the trouble of manually selecting and confirming storage. Personal information in radiography data is not kept forever, and is managed more properly and efficiently.

In a case in which the controller 111 receives a command to resend the request of entrusting save from the monitor 250 for external devices via the communicator 115, the controller 111 resends the request of entrusting save to an external device. Thus, an appropriate external device rather than the controller 111 itself may manage radiography data for which the response for entrusted save is not obtained. In addition to confirmation by the radiography control device 110, the PACS200 also confirms whether there is radiography data for which the transmitted response for entrusted save is not received. It prevents errors in confirmation. Radiography data for which the response for entrusted save is not obtained is surely identified.

In a case in which the controller 111 fails to obtain the response for entrusted save from the image management device 210, the controller 111 deletes personal information. The response for entrusted save may not be received as long as radiography data has been appropriately transferred. It reduces trouble. Personal information is immediately deleted and managed.

The controller 111 sets up to restrict disclosure of personal information included in radiography data 1122 kept in the storage 112. In a case in which data is designated as data not to be deleted and the radiography control device 110 keeps personal information for a standby period or longer, disclosure of the personal information is restricted until the personal information is deleted. For example, the recognition setting is performed. It prevents unauthorized users from unnecessarily accessing personal information. Personal information is not leaked.

The setting of restricting disclosure includes encryption of personal information. It prevents personal information from being stolen, regardless of how the data is accessed, unless recognition information is known. Personal information is managed more safely and is less likely to be leaked.

In a case in which radiography data 1122 kept in the storage 112 is not designated as data not to be deleted, the controller 111 deletes the radiography data including personal information. Thus, in a case in which radiographic image data does not need to be kept, the entire radiography data including personal information and the radiographic image data may be deleted at once. It saves memory space for radiographic image data. Even when many images are taken together, memory space is stably saved.

The communicator 115 wirelessly communicates with the image management device 210. The radiography control device 110, which controls the portable radiography device 150, is often brought to patient's rooms, etc. at various places. Use of wireless communication facilitates communication between the communicator 115 and the image management device 210. Communication condition often deteriorates depending on radio environment. Communication for radiography data, confirmation of completion of save, etc. is occasionally interrupted. Someone might access contents while the radiography control device 110 is stored. However, the radiography control device 110 appropriately confirms completion of data transfer as described above. Personal information is deleted or encrypted without being unnecessarily kept or disclosed. It effectively improves management of personal information.

The controller 111 monitors condition of wireless communication between the communicator 115 and the image management device 210. The controller 111 controls time to send radiography data 1122 and a request for confirming completion of save to the image management device 210 based on result of monitoring. Condition of wireless communication often deteriorates depending on radio environment as mentioned above. The controller 111 controls transmission of the radiography data 1122 and requests at appropriate times while monitoring communication condition. It reduces likelihood that transmitted information is not received. This eliminates troubles that it takes a long time for personal information to be deleted or that the same information is repeatedly sent. Personal information is less likely to be leaked.

The radiography system 100 according to the embodiment includes the above radiography control device 110 and the radiography device 150 that takes radiographic images. The radiography system 100 surely sends result of radiography to the PACS200 and manages personal information included in the result more properly.

The radiographic image processing method according to the embodiment includes:
controlling transmission of radiography data 1122 to the image management device 210 of the PACS200 and of a request for confirming completion of save of the radiography data 1122 by the communicator 115;
setting time to delete personal information included in the transmitted radiography data 1122 after confirming completion of save of the radiography data 1122; and
deleting the personal information in the storage 112 at the set time.

Thus, the radiography data 1122 is not kept for the maximum period the storage 112 can store, the period depending on the capacity of the storage 112. After an appropriate period passes, at least personal information is deleted. Therefore, personal information is managed more properly. Personal information is deleted after it is confirmed that radiography data is at least transferred and kept in the PACS200. It prevents necessary data from being accidentally deleted. Personal information is properly managed.

The controller 111 executes a program 1121 according to the embodiment. It more easily and surely prevents personal information from being leaked without redesigning the entire system or introducing special hardware configuration.

The present invention is not limited to the embodiments, and can be modified in various ways.

For example, in the embodiments, personal information and radiographic image data are deleted together when radiography data is not designated as data not to be deleted. However, radiographic image data may be deleted separately.

In the embodiments, after save is confirmed, the standby period to the deletion time is determined first. However, the standby period may be changed in the middle of the period. For example, if data becomes unnecessary before the standby period expires, change operation to omit the remaining standby period may be received.

In the embodiments, display contents are obtained at the set deletion time to determine whether to delete personal information. However, personal information may be deleted automatically at the deletion time without the determination. In that case, a predetermined warning or the like may be displayed in a case in which consistency is lost when display contents are switched.

The determination for deletion may not be based on display contents. Others such as status of data processing may be considered. In a case in which power supply to the radiography control device 110 is blocked or the controller 111 is not operating when the standby period expires, deletion operation may be performed in the first operation time of the controller 111 since the standby period expires.

The embodiments include examples for a case in which the response for entrusted save is not obtained for the request of entrusting save. In one example, personal information is immediately deleted. In another example, the request of entrusting save is transmitted again based on a predetermined trigger. These examples may be combined. For example, a time to delete personal information may be set when a predetermined number of the request of entrusting save is transmitted and no response is received.

The restriction of disclosure may be set at any time before personal information is deleted. For example, the restriction of disclosure may be set only in a case in which a user requests a setting of restricting disclosure through the operation interface 113.

In the above explanation, encryption is explained as an example of a setting of disclosure restriction. As a simpler processing, only display using a designated software may be restricted without encrypting the data.

In the embodiments, whether data is designated as data not to be deleted is determined, and radiographic image data is deleted along with personal information data in a case in which the data is not designated. However, in a case in which designation as data not to be deleted is not possible, radiographic image data and personal information data may be automatically deleted together without such determination after the standby period has passed.

In the embodiments, the radiography control device 110 as a radiographic image processing device and the image management device 210 directly exchange data. However, other devices, e.g., an image detector, may be put between the radiography control device 110 and the image management device 210. The image detector as a radiographic image processing device and the radiography control device 110 and/or the image management device 210 exchange radiography data and manage storage.

In the embodiments, the request from the monitor 250 is given as an example of a trigger for requesting retransmission of the request of entrusting save. However, the PACS200 may not have the monitor 250. In that case, the request of entrusting save is not resent in response to the trigger.

In the above explanation, the storage 112 having an auxiliary storage device, such as flash memory and/or an HDD, is given as an example of a computer-readable medium where the program 1121 for processing operation of the controller 111 according to the present invention is stored. However, the computer-readable medium is not limited to this. The computer-readable medium may be a portable recording medium such as CD-ROM or a DVD. A carrier wave may be applied to the present invention as a medium that provides data of the program of the invention via a communication line.

Specific configuration, contents and procedure of processing operation, etc. in the embodiments may be modified appropriately within the scope of the claims of the invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese patent application No. 2020-013256, filed on Jan. 30, 2020, is incorporated herein by reference in its entirety.

What is claimed is:

1. A radiographic image processing device, comprising:
a communicator that communicates with an external device;
a storage that stores radiography data; and
a controller,
wherein
the controller controls transmission of the radiography data and a request for confirming completion of save of the radiography data to the external device by the communicator,
the controller sets a deletion time to delete personal information included in the transmitted radiography data after confirming completion of save of the radiography data, and
the controller deletes the personal information in the storage at the set deletion time.

2. The radiographic image processing device according to claim 1, further comprising:
an operation interface,
wherein the controller sets the deletion time based on input operation received by the operation interface.

3. The radiographic image processing device according to claim 2, wherein a period from a time when completion of save is confirmed to the deletion time is longer than a predetermined shortest time limit.

4. The radiographic image processing device according to claim 2, wherein the controller deletes the personal information at a time when a predetermined condition is met after the deletion time.

5. The radiographic image processing device according to claim 4, further comprising:
a display that displays contents of the radiography data,
wherein
the controller determines, as the predetermined condition, whether change or selection of display or processing of the contents of the radiography data related to the personal information to be deleted is possible, and
in a case in which neither the change nor the selection is possible, the controller deletes the personal information.

6. The radiographic image processing device according to claim 5, wherein the case in which neither the change nor the selection is possible includes a period in which the controller switches display contents on the display.

7. The radiographic image processing device according to claim 1, wherein
the controller selects a piece of the radiography data for which notification of confirming completion of save is not obtained from the external device among pieces of the radiography data stored in the storage,
the controller resend the request for confirming completion of save for the selected piece of the radiography data to the external device.

8. The radiographic image processing device according to claim 7, wherein the controller sends the request for confirming completion of save again to the external device in a case in which the controller obtains a command to resend the request from an outside via the communicator.

9. The radiographic image processing device according to claim 1, wherein the controller deletes the personal information in a case in which the controller fails to obtain the notification of confirming completion of save from the external device.

10. The radiographic image processing device according to claim 1, wherein the controller performs a setting for restriction of disclosure of the personal information included in the radiography data kept in the storage.

11. The radiographic image processing device according to claim 10, wherein the setting includes encryption of the personal information.

12. The radiographic image processing device according to claim 1, wherein the controller deletes the radiography data including the personal information in a case in which the radiography data kept in the storage is not designated as data not to be deleted.

13. The radiographic image processing device according to claim 1, wherein the communicator wirelessly communicates with the external device.

14. The radiographic image processing device according to claim 13, wherein
the controller monitors condition of wireless communication with the external device, and
the controller controls a time to transmit the radiography data and the request for confirming completion of save to the external device based on result of monitoring.

15. A radiography system, comprising:
the radiographic image processing device according to claim 1; and
a radiography device that takes radiographic images.

16. A radiographic image processing method for a radiographic image processing device,
the device comprising:
a communicator that communicates with an external device; and
a storage that stores radiography data, and
the method comprising:
controlling transmission of the radiography data and a request for confirming completion of save of the radiography data to the external device by the communicator,
setting a deletion time to delete personal information included in the transmitted radiography data after confirming completion of save of the radiography data, and
deleting the personal information in the storage at the set deletion time.

17. A non-transitory computer readable medium storing a program that makes a computer of a radiographic image processing device perform processing,
the device comprising:
a communicator that communicates with an external device; and
a storage that stores radiography data, and
the processing comprising:
controlling transmission of the radiography data and a request for confirming completion of save of the radiography data to the external device by the communicator,
setting a deletion time to delete personal information included in the transmitted radiography data after confirming completion of save of the radiography data, and
deleting the personal information in the storage at the set deletion time.
